(19) United States
(12) Patent Application Publication
(10) Pub. No.: US 2007/0197593 A1
(43) Pub. Date: Aug. 23, 2007

(54) HYDROXYLAMINES AND DERIVATIVES FOR TREATMENT OF INFLAMMATORY CONDITIONS OF THE LIVER

(76) Inventors: William L. Matier, Hockessin, DE (US); Ghanshyam Patil, Lincoln University, PA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/677,455

(22) Filed: Feb. 21, 2007

Related U.S. Application Data
(60) Provisional application No. 60/775,456, filed on Feb. 22, 2006.

(51) Int. Cl. A61K 31/445; A61K 31/421

(52) U.S. Cl. 514/317; 514/327; 514/374

(57) ABSTRACT

Methods for the treatment and inhibition of hepatitis are disclosed. The methods utilize hydroxylamine compounds or ester derivatives thereof, administered to patients in an amount effective to treat or inhibit hepatitis.

### CROSS REFERENCE TO RELATED APPLICATIONS

This application claims priority to U.S. Provisional Application No. 60/775,456, filed Feb. 22, 2006; the disclosure of which is incorporated herein by reference in its entirety.

### FIELD OF THE INVENTION

The invention relates generally to the field of pharmacology. Specifically, the invention features methods for the treatment and inhibition of inflammatory conditions of the liver.

### BACKGROUND OF THE INVENTION

Various patents and other publications are referenced herein. The contents of each of these patents and publications are incorporated by reference herein, in their entireties. The entire contents of commonly-owned co-pending U.S. Publication Nos. 2004/0002461, 2005/0130906 and 2005/0131025 are incorporated by reference herein.

The liver is the largest gland in the body, and plays a vital role in, among other things, digestion, metabolism of carbohydrates, lipids, and proteins, storage of vitamins, minerals, and carbohydrates, production of blood clotting factors, destruction of bacteria in the blood, and detoxification of the body from endogenous and exogenous substances. Given the liver's broad spectrum of functions, diseases and pathologies of the liver can have wide-ranging systemic effects on the body. One such pathology is hepatitis.

Hepatitis is a generalized term for liver inflammation. Liver inflammation can be chronic or acute, and affects millions of individuals worldwide. The majority of these cases are classified as infectious hepatitis, meaning that they are capable of transmission to others. Infectious hepatitis is typically caused by viruses, most commonly the hepatitis A (HAV), hepatitis B (HBV), and hepatitis C(HCV) viruses. Other sources of infectious hepatitis include the hepatitis D virus (HDV), hepatitis E virus (HEV), and the putative hepatitis F and G viruses, as well as bacteria and other common viruses such as cytomegalovirus, Epstein-Barr virus, herpes simplex virus (HSV), and Varicella-Zoster virus, among others.

Hepatitis can also be classified as non-infectious, meaning that it is not capable of transmission to others. Examples of non-infectious hepatitis include alcoholic hepatitis, toxic/drug-induced hepatitis, autoimmune hepatitis, and granulomatus hepatitis. Alcoholic hepatitis can arise from excessive consumption of alcoholic beverages. Toxic/drug-induced hepatitis is the product of exposure to a toxin, drug, or chemical. Examples of common toxins that induce toxic/drug-induced hepatitis are aflatoxin or amanitin (from poisonous mushrooms). Autoimmune hepatitis results primarily from a cell-mediated (cytotoxic T cell) attack on liver tissue. Granulomatus hepatitis is characterized by an abnormal accumulation of white blood cells in the liver.

Cirrhosis of the liver results from damage to liver cells from toxins, inflammation, metabolic derangements and other causes. Damaged and dead liver cells are replaced by fibrous tissue, i.e., scarring of the liver. Liver cells regenerate in an abnormal pattern, forming nodules that are surrounded by the fibrous tissue. Grossly abnormal liver architecture eventually ensues, and this can lead to decreased blood flow to and through the liver, resulting in biochemical and functional abnormalities.

Oxidative stress is a pathology associated with both infectious and non-infectious hepatitis and can contribute to disease progression (Emerit I et al. (2005) Hepatogastroenterology 552:530-6; Pemberton P W et al. (2004) Biochim. Biophys. Acta. 1689:182-9, and Loguercio C et al. (2003) Free Radic. Biol. Med. 34:1-10). Antioxidants are a dietary means for combating oxidative stress. In fact, antioxidants have been demonstrated to exert a hepatoprotective effect (Amin A et al. (2005) Life Sci. 77:266-78), and have been proposed as a supplementary treatment for hepatitis (Dikici I et al. (2005) Clin. Biochem. 38:1141-4; Medina J et al. (2005) Drugs 65:2445-61; Melham A et al. (2005) J. Clin. Gastroenterol. 39:737-42; and, Peterhans E (1997) J. Nutr. 127:962 S-965S).

Nitroxides such as TEMPOL have been of greater interest because of their radical scavenging properties and exertion of an anti-inflammatory effect in various animal models of oxidative damage and inflammation. Nilsson et al. disclosed, in WO 88/05044, that nitroxides and their corresponding hydroxylamines are useful in prophylaxis and treatment of ischemic cell damage, presumably due to antioxidant effects. Paolini et al. (U.S. Pat. No. 5,981,548) disclosed N-hydroxylpiperidine compounds and their potential general utility in the treatment of pathologies arising from oxygen radicals and as foodstuff and cosmetic additives. Hsia et al. (U.S. Pat. Nos. 6,458,758, 5,840,701, 5,824,781, 5,817,632, 5,807,831, 5,804,561, 5,767,089, 5,741,893, 5,725,839 and 5,591,710) disclosed the use of stable nitroxides and hydroxylamines (e.g., TEMPOL and its hydroxylamine counterpart, TEMPOL-H), in combination with a variety of biocompatible macromolecules, to alleviate free radical toxicity in blood and blood components. Hahn et al. (1998, Int. J. Radiat. Oncol. Biol. Physics 42: 839-842; 2000, Free Rad. Biol. Med. 28: 953-958) reported on the in vivo radioprotection and effects on blood pressure of the stable free radical nitroxides and certain hydroxylamine counterparts.

Due to their comparative lack of toxicity, hydroxylamines are preferable to nitroxides as therapeutic agents. Published United States Patent Applications 2004/0002461, 2005/0130906 and 2005/0131025 to Matier and Patil disclose hydroxylamines and related compounds and their use in the treatment of a variety of ophthalmic conditions in which oxidative damage or inflammation are involved. Such compounds possess numerous advantageous qualities, including robust anti-inflammatory and antioxidant activities, as well as ocular permeability in some instances. However, while some nitroxides, e.g., TEMPOL, have demonstrated some anti-angiogenic activity, hydroxylamines heretofore have not been reported as possessing any efficacy to treat liver inflammation.

The various forms of hepatitis are typically treated with various chemotherapeutic regimens. However, many drugs currently used to treat hepatitis can exhibit undesirable side effects. Thus, newer drugs and methods of treatment with fewer or less severe side effects are desirable. Moreover it is also desirable to obtain drugs that can work synergistically with existing therapies to enhance their efficacy, or that can target the underlying molecular, biochemical, or physiological basis for hepatitis.

### SUMMARY OF THE INVENTION

The current disclosure features methods for treating or inhibiting hepatitis in a subject by administering to the subject a composition comprising a pharmaceutically acceptable carrier and a hydroxylamine compound or ester derivative thereof in a therapeutically sufficient amount effective to treat or inhibit hepatitis. In certain embodiments, the hydroxylamine compounds include Tempol-H, Tempo-H, and Oxano-H, and the derivatives of the hydroxylamine compounds have formula I: 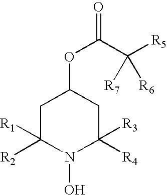
wherein:

R₁ and R₂ are, independently, H or C₁ to C₃alkyl;

R₃ and R₄ are, independently C₁ to C₃alkyl, or wherein R₁ and R₂, taken together, or R₃ and R₄, taken together, or R₁ and R₂, taken together and R₃ and R₄ taken together, are each cycloalkyl;

R₅ is H, OH, or C₁ to C₆alkyl;

R₆ is or C₁ to C₆alkyl, alkenyl, alkynyl, or substituted alkyl or alkenyl;

R₇ is C₁ to C₆alkyl, alkenyl, alkynyl, or substituted alkyl or alkenyl; or R₆ and R₇ taken together, or R₅, R₆, and R₇ taken together, form a carbocycle having from 3 to 7 atoms in the ring or form a heterocycle having from 3 to 7 atoms in the ring.

The inventive methods are effective to treat or inhibit hepatitis irrespective of the etiology. For example, the methods are effective to treat infectious hepatitis caused by the hepatitis A virus, hepatitis B virus, hepatitis C virus, hepatitis D virus, and hepatitis E virus, among others, as well as to treat non-infectious hepatitis such as alcoholic hepatitis, toxic/drug-induced hepatitis, autoimmune hepatitis, and granulomatus hepatitis. The infectious or non-infectious hepatitis can be acute or chronic. The methods can be used in any animal, and preferably are used in mammals, and most preferably are used in humans.

In some aspects of the invention, the compositions of the invention are used synergistically with other anti-hepatic or anti-inflammatory agents, or with antioxidants.

Other features and advantages of the invention will be understood by reference to the drawings, detailed description, and examples that follow.

### BRIEF DESCRIPTION OF THE DRAWINGS

FIG. 1 shows histopathological examination of liver tissue from rat 2801. Mild subacute hepatitis is apparent. Magnification is at 20×.

FIG. 2 shows histopathological examination of liver tissue from rat 2803. Only minimal subacute hepatitis is observed. Magnification is at 20×.

FIG. 3 shows histopathological examination of liver tissue from rat 2809. The liver is normal, showing no hepatitis. Magnification is at 20×.

FIG. 4 shows histopathological examination of liver tissue from rat 2817. The liver is normal, showing no hepatitis. Magnification is at 20×.

FIG. 5 shows histopathological examination of liver tissue from rat 2825. The liver is normal, showing no hepatitis. Magnification is at 20×.

FIG. 6 shows histopathological examination of liver tissue from rat 2861. Mild subacute hepatitis is apparent. Magnification is at 20×.

FIG. 7 shows histopathological examination of liver tissue from rat 2860. Only minimal subacute hepatitis is observed. Magnification is at 20×.

FIG. 8 shows histopathological examination of liver tissue from rat 2865. The liver is normal, showing no hepatitis. Magnification is at 20×.

FIG. 9 shows histopathological examination of liver tissue from rat 2873. The liver is normal, showing no hepatitis. Magnification is at 20×.

FIG. 10 shows histopathological examination of liver tissue from rat 2887. The liver is normal, showing no hepatitis. Magnification is at 20×.

### DETAILED DESCRIPTION OF ILLUSTRATIVE EMBODIMENTS

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

The following abbreviations may be used in the specification and examples: HAV, hepatitis A virus; HBV, hepatitis B virus; HCV, hepatitis C virus, HDV, hepatitis D virus; HEV, hepatitis E virus; HFV, hepatitis F virus; HGV, hepatitis G virus.

The terms “treating” or “treatment” refer to any success or indicia of success in the attenuation or amelioration of an injury, pathology or condition, including any objective or subjective parameter such as abatement, remission, diminishing of symptoms or making the injury, pathology, or condition more tolerable to the patient, slowing in the rate of degeneration or decline, making the final point of degeneration less debilitating, improving a subject's physical or mental well-being, or prolonging the length of survival. The treatment or amelioration of symptoms can be based on objective or subjective parameters; including the results of a physical examination, neurological examination, and/or psychiatric evaluations.

“Effective amount” or “therapeutically effective amount” are used interchangeably herein, and refer to an amount of a compound, material, or composition, as described herein effective to achieve a particular biological result. Such results may include, but are not limited to, the treatment of both infectious and non-infectious hepatitis in a subject, as determined by any means suitable in the art.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

“Hepatitis” refers to any clinically significant inflammation of the liver or biliary system, regardless of etiology. “Acute hepatitis” refers to any short term (less than six months) or initial-stage liver inflammation, such as the initial stages of hepatitis virus infection. “Chronic hepatitis” refers to any inflammation of the liver persisting six months or longer. “Infectious hepatitis” refers to any inflammation of the liver that can be transmitted to others. Typically, infectious hepatitis is caused by a microorganism such as a virus (e.g., HAV, HBV, HCV, HDV, HEV, HFV, HGV, cytomegalovirus, Epstein-Barr virus, herpes simplex virus (HSV), and Varicella-Zoster virus, etc.), bacteria, protozoan, or yeast. “Non-infectious hepatitis” refers to any inflammation of the liver that cannot be transmitted to others, such as alcoholic hepatitis, autoimmune hepatitis, toxic/drug induced hepatitis, and granulomatus hepatitis, and the like.

The terms “biliary system” or “biliary tissue” refer to the organs and duct system that create, transport, store, and release bile into the small intestine. The term encompasses the liver, gallbladder, and bile ducts: the cystic duct, hepatic duct, common hepatic duct, common bile duct, and pancreatic duct.

“Etiology” means the cause or origin of a disease, disorder, or pathology.

The present invention provides compositions and methods for the treatment or inhibition of hepatitis. The methods comprise administration of compositions comprising a pharmaceutically acceptable carrier and a hydroxylamine compound or ester derivative thereof in a therapeutically sufficient effective to treat, inhibit, or slow the progression of hepatitis.

The invention further provides hydroxylamine compounds, including tempol-H, tempo-H, and oxano-H, and any pharmaceutically acceptable salts, analogs, homologs, conjugates, and derivatives thereof, in the manufacture of a medicament for the treatment or inhibition of hepatitis, including acute and chronic hepatitis and infectious and non-infectious hepatitis.

Preferred examples of the type of hydroxylamine compounds suitable for use in the present invention are TEMPOL-H (TPH, the hydroxylamine reduced form of the nitroxide 4-hydroxy-2,2,6,6-tetramethylpiperidin-1-yloxy), TEMPO-H (the hydroxylamine reduced form of the nitroxide 2,2,6,6-tetramethylpiperidin-1-yloxy) and OXANO-H (2-Ethyl-2,4,4-trimethyl-oxazolidin-3-ol), which is the reduced form of OXANO, 2-ethyl-2,4,4-trimethyloxazolidin-3-yloxy). Other hydroxylamine compounds suitable for use in the present invention include, but are not limited to, those disclosed by Hahn et al. (1998, supra; 2000, supra), Samuni et al. (2001, supra); and in U.S. Pat. No. 5,981,548 to Paolini, et al. (disclosing certain N-hydroxylpiperidine esters and their use as antioxidants in a number of contexts); U.S. Pat. No. 4,404,302 to Gupta et al. (disclosing the use of certain N-hydroxylamines as light stabilizers in plastics formulations); U.S. Pat. No. 4,691,015, to Behrens et al. (describing hydroxylamines derived from hindered amines and the use of certain of them for the stabilization of polyolefins); the hydroxylamine compounds disclosed in the several aforementioned U.S. patents to Hsia et al.; and the hydroxylamine counterparts of the nitroxides disclosed in U.S. Pat. Nos. 5,462,946 and 6,605,619 to Mitchell et al., namely, (1) compounds of the formula R₃—N(R₄)(R₅) wherein R₃ is —OH and R₄ and R₅ combine together with the nitrogen to form a heterocycle group, or wherein R₄ and R₅ themselves comprise a substituted or unsubstituted cyclic or heterocyclic group; (2) metal-independent hydroxylamines of formula R₃—N(R₄)(R₅) wherein R₃ is —OH and R₄ and R₅, together with the nitrogen atom to which they are bonded, form a 5- or 6-membered heterocyclic group, which, in addition to said nitrogen atom, comprises one or more heteroatoms selected from the group consisting of oxygen, nitrogen and sulfur, or R₄ and R₅, separately, each comprise a substituted or unsubstituted 5- or 6-membered cyclic group or a substituted or unsubstituted 5- or 6-membered heterocyclic group, which comprises one or more heteroatoms selected from the group consisting of oxygen, nitrogen and sulfur; or (3) oxazolidine compounds of the formula:

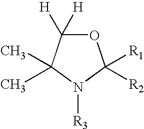

wherein R₁ is —CH₃ and R₂ is —C₂H₅, —C₃H₇, —C₄H₉, —C₅H₁₁, —C₆H₁₃, —CH₂CH(CH₃)₂, —CHCH₃C₂H₅, or —(CH₂)₇CH₃, and R₃ is —OH, or wherein R₁ and R₂ together form spirocyclopentane, spirocyclohexane, spirocycloheptane, spirocyclooctane, 5-cholestane or norbornane; and pharmaceutically acceptable salts of any of the above-listed compounds. Insofar as is known, the above-referenced compounds have not been used heretofore for treating or inhibiting hepatitis.

Ester derivatives of hydroxylamines suitable for use in the present invention comprise compounds of formula I or their pharmaceutically acceptable salts, examples of which are described in detail in U.S. Published Application 2004/0002461: 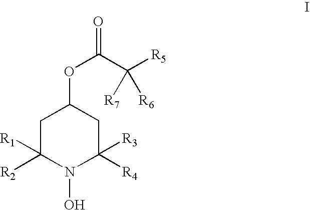
wherein:

R₁ and R₂ are, independently, H or C₁ to C₃alkyl;

R₃ and R₄ are, independently C₁ to C₃alkyl, or wherein R₁ and R₂, taken together, or R₃ and R₄, taken together, or R₁ and R₂, taken together and R₃ and R₄ taken together, are each cycloalkyl;

R₅ is H, OH, or C₁ to C₆alkyl;

R₆ is or C₁ to C₆alkyl, alkenyl, alkynyl, or substituted alkyl or alkenyl;

R₇ is C₁ to C₆alkyl, alkenyl, alkynyl, or substituted alkyl or alkenyl; or R₆ and R₇ taken together, or R₅, R₆, and R₇ taken together, form a carbocycle having from 3 to 7 atoms in the ring or form a heterocycle having from 3 to 7 atoms in the ring.

The methods of the present invention may also utilize compositions comprising a pharmaceutically acceptable carrier or diluent and a hydroxylamine compound having an N-hydroxy piperidine portion bound to a solubility modifying portion, the compound having a solubility in water at 25° C. of at least about 0.25% by weight and a water/n-octanol partition coefficient at 25° C. of at least about 5. The composition may have the N-hydroxy piperidine portion cleavable from the compound under conditions found in biological tissues, such as found in the eye. The N-hydroxy piperidine portion may be cleaved enzymatically. The compositions may also exist wherein the N-hydroxy piperidine portion is 1-oxyl-4-hydroxy-2,2,6,6-tetramethylpiperidyl.

The term C₁ to Cₙ alkyl, alkenyl, or alkynyl, in the sense of this invention, means a hydrocarbyl group having from 1 to n carbon atoms in it, wherein n is an integer from 1 to about 20, preferably 1 to about 10, yet more preferably, 1 to about 6, with from 1 to about 3 being even more preferred. The term thus comprehends methyl, ethyl, n-propyl, iso-propyl, n-butyl, sec-butyl, iso-butyl, tert-butyl, and the various isomeric forms of pentyl, hexyl, and the like. Likewise, the term includes ethenyl, ethynyl, propenyl, propynyl, and similar branched and unbranched unsaturated hydrocarbon groups of up to n carbon atoms. As the context may admit, such groups may be functionalized such as with one or more hydroxy, alkoxy, alkylthio, alkylamino, dialkylamino, aryloxy, arylamino, benzyloxy, benzylamino, heterocycle, or YCO-Z, where Y is O, N, or S and Z is alkyl, cycloalkyl, heterocycle, or aryl substitutent.

The term carbocycle defines cyclic structures or rings, wherein all atoms forming the ring are carbon. Exemplary of these are cyclopropyl, cyclobutyl, cyclopentyl, cyclohexyl, cycloheptyl, etc. Cyclopropyl is one preferred species. Heterocycle defines a cyclic structure where at least one atom of the ring is not carbon. Examples of this broad class include furan, dihydrofuran, tetrahydrofuran, pyran, oxazole, oxazoline, oxazolidine, imidazole and others, especially those with an oxygen atom in the ring. Five, six and seven membered rings with at least one oxygen or nitrogen atom in the ring are preferred heterocycles. Furanyl and tetrahydrofuranyl species are among those preferred.

It is preferred for certain embodiments that each of R₁ through R₄ be lower alkyl that is C₁ to C₃ alkyl. Preferably, all these groups are methyl for convenience in synthesis and due to the known efficacy of moieties having such substitution at these positions. However, other substitutents may be used as well.

In certain embodiments, compounds are employed where R₆ is C₁ to C₆ alkyl substituted with at least one C₁ to C₆ alkoxy or benzyloxy group. Preferred among these are compounds having ethoxy or benzyloxy substitutents. Among preferred compounds are those where each of R₁ through R₄ is methyl, R₅ is H or methyl, R₆ is methyl substituted with benzyloxy or C₁ to C₆ alkoxy, and R₇ is methyl or where R₆ and R₇ form a cyclopropyl group as well as the compound in which each of R₁ through R₄ is methyl, R₅ is methyl, R₆ is ethoxy or benzyloxy methyl, and R₇ is methyl. An additional preferred compound is one in which each of R₁ through R₄ is methyl, R₅ is methyl, R₆ is hydroxymethyl, and R₇ is methyl.

Other useful compounds are those wherein each of R₁ through R₄ is methyl, and R₅, R₆, and R₇ form a furanyl group, or in which R₆ and R₇ form a tetrahydrofuranyl group. The compound where R₁ through R₄ is methyl, R₅ is H and, R₆ and R₇ form a cyclopropyl ring is a further preferred. Examples of compounds useful in the methods of the present invention include, but are not limited to those described in U.S. Patent Publication No. US 2004/0002461A1, such as 1-oxyl-4-(3′-ethoxy-2′,2′-dimethyl)propanecarbonyloxy-2,2,6,6-tetramethylpiperidine; 1-hydroxy-4-(3′-ethoxy-2′,2′-dimethyl)propanecarbonyloxy-2,2,6,6-tetramethylpiperidine hydrochloride; 1-oxyl-4-cyclopropanecarbonyloxy-2,2,6,6-tetramethylpiperidine; 1-hydroxy-4-cyclopropanecarbonyloxy-2,2,6,6-tetramethylpiperidine hydrochloride; 1-oxyl-4-(3′-benzyloxy-2′,2′-dimethyl)propanecarbonyloxy-2,2,6,6-tetramethylpiperidine; 1-hydroxy-4-(3′-benzyloxy-2′,2′-dimethyl)propanecarbonyloxy-2,2,6,6-tetramethylpiperidine hydrochloride; 1-hydroxy-4-(3′-hydroxy-2′,2′-dimethyl)propanecarbonyloxy-2,2,6,6-tetramethylpiperidine hydrochloride; 1-oxyl-4-(1-methyl-cyclopropane) carbonyloxy-2,2,6,6-tetramethylpiperidine; 1-hydroxy-4-(1-methyl-cyclopropane)carbonyloxy-2,2,6,6-tetramethylpiperidine hydrochloride; 1-oxyl-4-(2-furan)carbonyloxy-2,2,6,6-tetramethylpiperidine; 1-hydroxy-4-(2′-furan)carbonyloxy-2,2,6,6-tetramethylpiperidine hydrochloride; 1-oxyl-4-(3′-tetrahydrofuran)carbonyloxy-2,2,6,6-tetramethylpiperidine; 1-hydroxy-4-(3′-tetrahydrofuran)carbonyloxy-2,2,6,6-tetramethylpiperidine hydrochloride. 1-Hydroxy-4-cyclopropanecarbonyloxy-2,2,6,6-tetramethylpiperidine hydrochloride, referred to herein as Compound 1, is particularly preferred.

While not wishing to be bound by theory, Applicants believe that Compound 1 (compound of formula 1, wherein R¹, R², R³, and R⁴ are methyl, R⁵ is H, and R⁶ and R⁷ taken together form a cyclopropane ring) and the other compounds of formula I are believed exert their anti-inflammatory and other therapeutic effects in two ways. First, the ester compounds are hydrolyzed in situ to form hydroxylamine components that exert therapeutic activity. Second, the esterified compounds themselves possess antioxidant activity, and therefore may possess anti-inflammatory activity, thereby supporting the therapeutic efficacy of pharmaceutical preparations comprising the compounds.

In connection with the first basis for activity of the compounds of formula I, i.e., cleavage to liberate hydroxylamine components, numerous esterases are known to be present in various tissues and organs of the body. The specific esterase(s) that cleaves the esters of the present series need not be identified in order to practice the invention.

The compositions can be prepared in a wide variety of dosage forms according to any means suitable in the art for preparing a given dosage form. Pharmaceutically acceptable carriers can be either solid or liquid. Non-limiting examples of solid form preparations include powders, tablets, pills, capsules, lozenges, cachets, suppositories, dispersible granules, and the like. A solid carrier can include one or more substances which may also act as diluents, flavoring agents, buffering agents, binders, preservatives, tablet disintegrating agents, or an encapsulating material. Suitable solid carriers include magnesium carbonate, magnesium stearate, talc, sugar, lactose, pectin, dextrin, starch, gelatin, acacia, tragacanth, methylcellulose, sodium carboxymethyl-cellulose, polyethylene glycols, vegetable oils, agar, a low melting wax, cocoa butter, and the like. Non-limiting examples of suitable disintegrating agents include the cross-linked polyvinylpyrrolidone, agar, or alginic acid or a salt thereof such as sodium alginate. Non-limiting examples of liquid form preparations include solutions, suspensions, syrups, slurries, and emulsions. Suitable liquid carriers include any suitable organic or inorganic solvent, for example, water, alcohol, saline solution, physiological saline, buffered saline, dextrose solution, water propylene glycol solutions, and the like, preferably in sterile form.

The compositions can be formulated and administered to the subject as pharmaceutically acceptable salts. Non-limiting examples of pharmaceutically acceptable salts include acid addition salts such as those containing hydrochloride, sulfate, phosphate, sulfamate, acetate, citrate, lactate, tartrate, methanesulfonate, ethanesulfonate, benzenesulfonate, p-toluenesulfonate, cyclohexylsulfamate and quinate. Such salts can be derived using acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid, according to means known and established in the art.

Aqueous solutions can be prepared by dissolving the active component in water and adding suitable colorants, flavors, stabilizing and thickening agents as desired. Aqueous suspensions can also be made by dispersing the finely divided active component in water with viscous material, such as natural or synthetic gums, resins, methylcellulose, sodium carboxymethylcellulose, and other well-known suspending agents.

Solid forms can be prepared according to any means suitable in the art. For example, capsules are prepared by mixing the composition with a suitable diluent and filling the proper amount of the mixture in capsules. Tablets are prepared by direct compression, by wet granulation, or by dry granulation. Their formulations usually incorporate diluents, binders, lubricants and disintegrators as well as the compound. Nonlimiting examples of diluents include various types of starch, cellulose, crystalline cellulose, microcrystalline cellulose, lactose, fructose, sucrose, mannitol, kaolin, calcium phosphate or sulfate, inorganic salts such as sodium chloride and powdered sugar. Powdered cellulose derivatives are also useful. Nonlimiting examples of tablet binders include starch, gelatin and sugars such as lactose, fructose, glucose and the like. Natural and synthetic gums can also be used, including acacia, alginates, methylcellulose, polyvinylpyrrolidine and the like. Polyethylene glycol, ethylcellulose and waxes can also serve as binders.

A lubricant can be used in a tablet formulation to prevent the tablet and punches from sticking in the die. The lubricant can be chosen from such slippery solids as talc, magnesium and calcium stearate, stearic acid and hydrogenated vegetable oils. Tablet disintegrators are substances which swell when-wetted to break up the tablet and release the compound, and include starches such as corn and potato starches, clays, celluloses, algins and gums, methylcellulose, agar, bentonite, wood cellulose, powdered natural sponge, cation-exchange resins, alginic acid, guar gum, citrus pulp, carboxymethyl cellulose, and sodium lauryl sulfate. Tablets can be coated with sugar as a flavor and sealant, or with film-forming protecting agents to modify the dissolution properties of the tablet. The compounds may also be formulated as chewable tablets, by using large amounts of pleasant-tasting substances such as mannitol in the formulation, as is now well-established in the art.

Also included are liquid formulations and solid form preparations which are intended to be converted, shortly before use, to liquid form preparations. Such liquid forms include solutions, suspensions, syrups, slurries, and emulsions. Liquid preparations may be prepared by conventional means with pharmaceutically acceptable additives such as suspending agents (e.g., sorbitol syrup, cellulose derivatives or hydrogenated edible fats or oils); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). These preparations may contain, in addition to the active agent, colorants, flavors, stabilizers, buffers, artificial and natural sweeteners, dispersants, thickeners, solubilizing agents, and the like. The compositions may be in powder form for constitution with a suitable vehicle such as sterile water, saline solution, or alcohol, before use.

Compositions for use in topical administration include, e.g., liquid or gel preparations suitable for penetration through the skin such as creams, liniments, lotions, ointments or pastes, and drops suitable for delivery to the eye, ear or nose.

In some embodiments, the present compositions include creams, drops, liniments, lotions, ointments and pastes are liquid or semi-solid compositions for external application. Such compositions may be prepared by mixing the active ingredient(s) in powdered form, alone or in solution or suspension in an aqueous or non-aqueous fluid with a greasy or non-greasy base. The base may comprise complex hydrocarbons such as glycerol, various forms of paraffin, beeswax; a mucilage; a mineral or edible oil or fatty acids; or a macrogel. Such compositions may additionally comprise suitable surface active agents such as surfactants, and suspending agents such as agar, vegetable gums, cellulose derivatives, and other ingredients such as preservatives, antioxidants, and the like.

The compositions can also be formulated for injection into the subject. For injection, the compositions of the invention can be formulated in aqueous solutions such as water or alcohol, or in physiologically compatible buffers such as Hanks's solution, Ringer's solution, or physiological saline buffer. The solution may contain formulatory agents such as suspending, stabilizing and/or dispersing agents. Injection formulations may also be prepared as solid form preparations which are intended to be converted, shortly before use, to liquid form preparations suitable for injection, for example, by constitution with a suitable vehicle, such as sterile water, saline solution, or alcohol, before use.

The compositions may also be formulated in sustained release vehicles or depot preparations. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Liposomes and emulsions are well-known examples of delivery vehicles suitable for use as carriers for hydrophobic vehicles or carriers for hydrophilic drugs.

The compositions may further include one or more antioxidants. Exemplary reducing agents include mercaptopropionyl glycine, N-acetylcysteine, β-mercaptoethylamine, glutathione, ascorbic acid and its salts, sulfite, or sodium metabisulfite, or similar species. In addition. antioxidants can also include natural antioxidants such as vitamin E, C, leutein, xanthine, beta carotene and minerals such as zinc and selenium.

Administration of the compositions can be by infusion or injection (intravenously, intraarterially, intramuscularly, intracutaneously, subcutaneously, intrathecal, intraduodenally, intraperitoneally, and the like). The compositions can also be administered intranasally, vaginally, rectally, orally, topically, or transdermally. Preferably, the compositions are administered orally. Administration can be at the direction of a physician.

For buccal administration, the compositions may take the form of tablets, troche or lozenge formulated in conventional manner. Compositions for oral or buccal administration, may be formulated to give controlled release of the active compound. Such formulations may include one or more sustained-release agents known in the art, such as glyceryl mono-stearate, glyceryl distearate and wax.

Various alternative pharmaceutical delivery systems may be employed. Non-limiting examples of such systems include liposomes and emulsions. Certain organic solvents such as dimethylsulfoxide also may be employed. Additionally, the compounds may be delivered using a sustained-release system, such as semipermeable matrices of solid polymers containing the therapeutic agent. The various sustained-release materials available are well known by those skilled in the art. Sustained-release capsules may, depending on their chemical nature, release the compounds over a range of several days to several weeks to several months.

The compositions utilized in accordance with the inventive methods may contain more than one hydroxylamine compound. In some embodiments, two or more hydroxylamines are administered simultaneously. In other embodiments, they are administered sequentially.

The compositions of the invention for treating hepatitis may also be co-administered with other well known therapeutic agents that are selected for their particular usefulness against the condition that is being treated. For example, such therapeutic agents can be pain relievers, anti-inflammatory agents, antibiotics, anti-viral agents, anti-cirrhotic, or other known agents that treat or inhibit hepatitis.

In general, anti-hepatic agents can be any known inhibitor or down regulator of inflammation and the inflammatory response. Non-limiting examples of anti-hepatic agents include COX-2 inhibitors, lamivudine, interferon alpha-2a, interferon alpha-2b, interferon-n3, interferon-alfacon, peginterferon alpha-2a, peginterferon alpha-2b, and ribavirus. Non-limiting examples of anti-inflammatory agents include non-steroidal anti-inflammatory drugs (NSAIDs), corticosteroids, lactoferrin, and herbs and herbal extracts such as Silybum marianum (milk thistle), Phyllanthus, glycyrrhizin (licorice root extract), Picrorhiza kurroa, Cudrania cochinchinensis var. gerontogea, Bidens pilosa, Glossogyne tenuifolia, scoparone from Artemisia capillaries, and Sargassum polycystum, among others. (See, Ishikado A et al. (2005) Biol. Pharm. Bull. 28:1717-21; Dhiman R K et al. (2005) Dig. Dis. Sci. 50:1807-12; We M J et al. (2005) Life Sci. 76:1135-46; Jang S et al. (2005) Arch. Pharm. Res. 28:203-8; Raghavendran H R (2005) Mol. Cell. Biochem. 276:89-96; Abajo C et al. (2004) J. Ethnopharmacol. 93:319-23; Lin C C et al. (1999) Am. J. Chin. Med. 27:227-39; and, Luper S (1998) Altern. Med. Rev. 3:410-21).

The administration of these additional compounds may be simultaneous with the administration of the hydroxylamine compounds, or may be administered in tandem, either before or after the administration of the hydroxylamine compounds, as necessary. Any suitable protocol may be devised whereby the various compounds to be included in the combination treatment are administered within minutes, hours, days, or weeks of each other. Repeated administration in a cyclic protocol is also contemplated to be within the scope of the present invention.

To treat a subject afflicted with hepatitis, a therapeutically effective amount of a composition comprising a pharmaceutically acceptable carrier and at least one hydroxylamine compound or ester derivative thereof is administered to the subject. A therapeutically effective amount will provide a clinically significant decrease in localized or systemic inflammation of the liver or biliary tissue, or the inhibition of the onset or progression of hepatitis, and the like. The compositions are effective to treat chronic and acute hepatitis, as well as infectious and non-infectious hepatitis, and can be administered to any animal, particularly mammals such as dogs, cats, rats, mice, rabbits, horses, pigs, cows, sheep, and donkeys, and are preferably administered to humans.

The effective amount of the composition may be dependent on any number of variables, including without limitation, the species, breed, size, height, weight, age, overall health of the subject, the type of formulation, the mode or manner or administration, or the severity of the hepatitis or other related condition. The appropriate effective amount can be routinely determined by those of skill in the art using routine optimization techniques and the skilled and informed judgment of the practitioner and other factors evident to those skilled in the art. Preferably, a therapeutically effective dose of the compounds described herein will provide therapeutic benefit without causing substantial toxicity to the subject.

Toxicity and therapeutic efficacy of agents or compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Agents or compositions which exhibit large therapeutic indices are preferred. The dosage of such agents or compositions lies preferably within a range of circulating concentrations that include the ED50 with little or no toxicity. The dosage may vary within this range depending upon the dosage form employed and the route of administration utilized.

For the compositions used in the inventive methods, the therapeutically effective dose can be estimated initially from in vitro assays such as cell culture assays. For example, a dose can be formulated in animal models to achieve a circulating plasma concentration range that includes the IC50 as determined in cell culture (i.e., the concentration of the composition which achieves a half-maximal inhibition of the osteoclast formation or activation). Such information can be used to more accurately determine useful doses in a specified subject such as a human. The treating physician can terminate, interrupt, or adjust administration due to toxicity, or to organ dysfunctions, and can also adjust treatment as necessary if the clinical response was not adequate in order to improve the clinical response.

In the inventive methods, the compositions comprise a concentration of a hydroxylamine compound in a range of about 0.01% to about 90% of the dry matter weight of the composition. A daily dose range of about 0.01 mg/kg to about 100 mg/kg of the weight of the subject is preferred. Preferably, the daily dose ranges from about 0.1 mg/kg to about 50 mg/kg of the weight of the subject. More preferably, the daily dose ranges from about 1 mg/kg to about 10 mg/kg of the weight of the subject.

In preferred embodiments, administration of the compositions comprising hydroxylamine compounds to a subject will achieve a concentration of the hydroxylamine component in the range of about 0.1 μM to about 10 mM in the tissues and fluids of the subject, preferably in the liver or biliary tissue. In some embodiments, the range is from 1 μm to 5 mM, in other embodiments the range is about 10 μM to 2.5 mM. In still other embodiments, the range is about 50 μM to 1 mM. Most preferably the range of hydroxylamine concentration will be from 1 to 100 μM in the tissues and fluids of the subject, preferably in the liver. In embodiments that include a reducing agent, either within the formulation or administered separately, the concentration of the reducing agent will be from 1 μM to 5 mM in the tissues and fluids of the subject to which the composition is administered, particularly in the liver, preferably in the range of 10 μM to 2 mM. The concentrations of the components of the composition are adjusted appropriately to the route of administration, by typical pharmacokinetic and dilution calculations, to achieve such local concentrations.

Treatment can be initiated with smaller dosages that are less than the optimum dose of the hydroxylamine compound, followed by an increase in dosage over the course of the treatment until the optimum effect under the circumstances is reached. If needed, the total daily dosage may be divided and administered in portions throughout the day.

For effective treatment of hepatitis, one skilled in the art may recommend a dosage schedule and dosage amount adequate for the subject being treated. It may be preferred that dosing occur one to four times daily for as long as needed. The dosing may occur less frequently if the compositions are formulated in sustained delivery vehicles. The dosage schedule may also vary depending on the active drug concentration, which may depend on the needs of the subject.

Techniques and formulations for administering above-described compositions may be found in Remington's Pharmaceutical Sciences, Meade Publishing Col., Easton, Pa., 20^{th} edition (2003).

The following examples are provided to describe the invention in greater detail. They are intended to illustrate, not to limit, the invention.

### EXAMPLE 1

### Anti-Inflammatory Effects of Hydroxylamines in Rat Livers

Materials and Methods:

Potential toxicity of Compound 1-HCl was evaluated by intravenous administration to Sprague Dawley rats for 28 days. This study was conducted in compliance with FDA Good Laboratory Practice Standards, 21 CFR 58 and in accordance with OECD Principles of Good Laboratory Practice.

One hundred twelve Sprague Dawley—HsD: SD rats were randomly divided into eight groups and treated with Compound 1 (“test article”) or vehicle, by intravenous administration via the lateral tail vein (slow bolus) (Table 1).

TABLE 1
Group Designation and Dosage Levels

| | | | Dose | | | |
|---|---|---|---|---|---|---|
| | | Dose | Volume | Concentration | Number of Animals | |
| Group | Treatment | (mg/kg/day) | (ml/kg/day) | (mg/ml) | Males | Females |

Toxicology Groups

| | | | | | | |
|---|---|---|---|---|---|---|
| 1 | Vehicle | 0 | 10 | 0 | 8 | 8 |
| 2 | Cpd 1-HCl | 1.0 | 10 | 0.1 | 8 | 8 |
| 3 | Cpd 1-HCl | 2.5 | 10 | 0.25 | 8 | 8 |
| 4 | Cpd 1-HCl | 7.5 | 10 | 0.75 | 8 | 8 |

Toxicokinetic Groups*

| | | | | | | |
|---|---|---|---|---|---|---|
| 5 | Vehicle | 0 | 10 | 0 | 6 | 6 |
| 6 | Cpd 1-HCl | 1.0 | 10 | 0.1 | 6 | 6 |
| 7 | Cpd 1-HCl | 2.5 | 10 | 0.25 | 6 | 6 |
| 8 | Cpd 1-HCl | 7.5 | 10 | 0.75 | 6 | 6 |

*Toxicokinetic groups are not discussed in this example.

Animals in Groups 1-4 were subjected to a complete necropsy examination. Protocol-specified necropsy procedures included the recording of organ weights (adrenals, brain, hearts, kidneys, liver, testes, ovaries, spleen, thyroids, parathyroids), examination of the external body surface, examination of all orifices, and examination of the cranial, thoracic, and abdominal cavities and their contents. Post-fixation weights for the thyroids/parathyroids were recorded. The following protocol-specified tissues were collected at necropsy: animal identification (not evaluated), abnormalities (gross findings), adrenals, aorta, brain, cecum, cervix, colon, duodenum, epidiymides, esophagus, eye with optic nerve, femur with articular surface, heart, ileum, injection site, jejunum, kidneys, lacrimal glands (exorbital), larynx, liver, lung with mainstem bronchus, mammary gland, mandibular lymph nodes, mesenteric lymph nodes, ovaries, pancreas, pituitary, prostate, rectum, salivary glands, sciatic nerve, seminal vesicles, skeletal muscle (thigh), skin, spinal cord (cervical, midthoracis, lumbar), spleen, sternum with bone marrow, stomach, testes, thymus, thyroid/parathyroid, tongue, trachea, urinary bladder, uterus, and vagina.

On day 29, animals were weighed and sacrificed by CO₂ inhalation. Protocol-specific tissues were preserved in 10% neutral buffered formalin (NBF). Protocol-requires tissues were embedded in paraffin, sectioned at approximately 5 microns, and processed to slides stained with hematoxylin and eosin (H&E). Histopathological evaluation was performed by a veterinary pathologist on all rats in Group 1 and Group 4 sacrificed on day 29, as well as for all early deaths. Livers for animals in Groups 2 and 3 were evaluated microscopically.

Results:

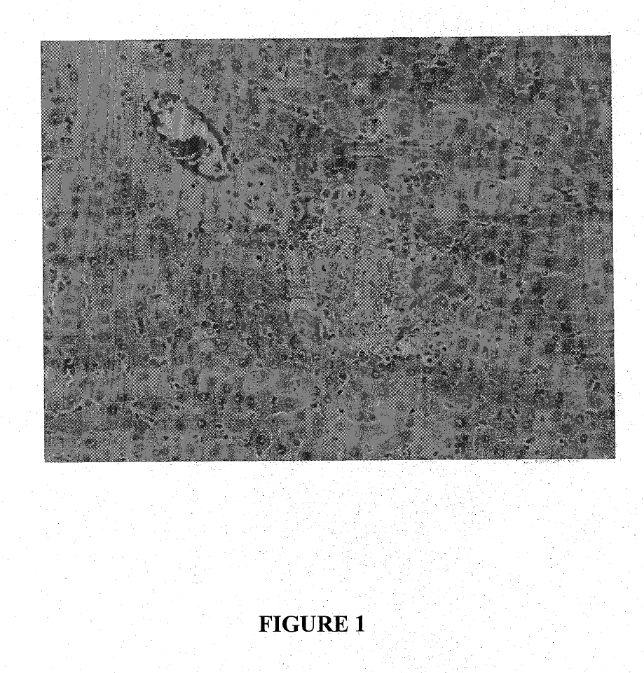

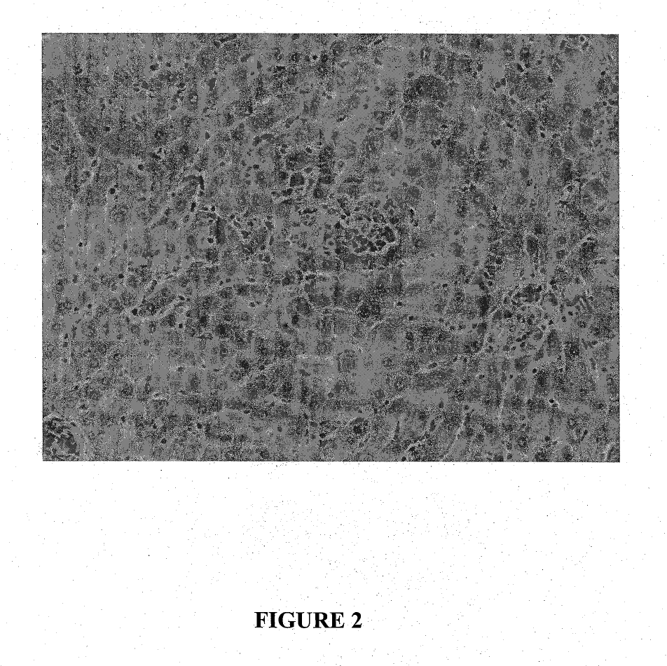

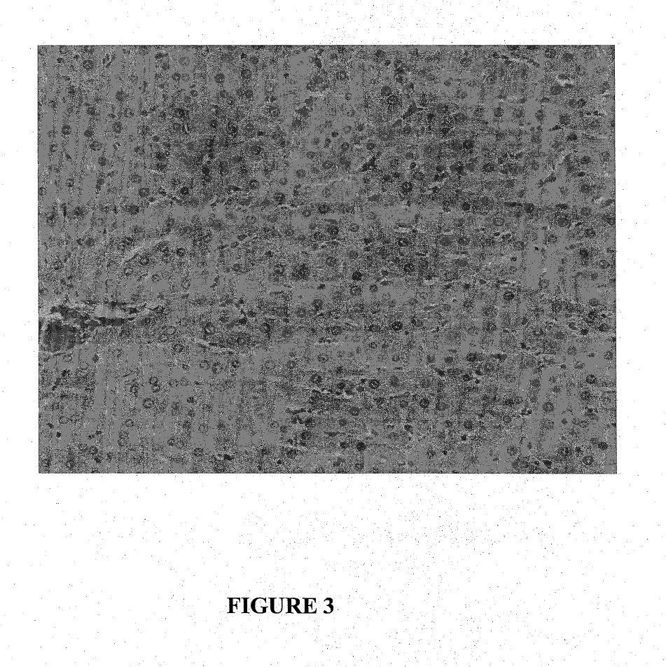

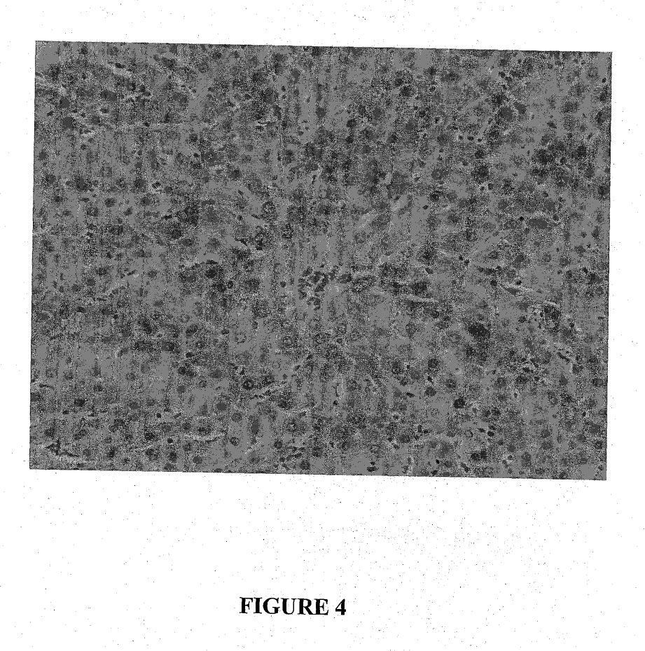

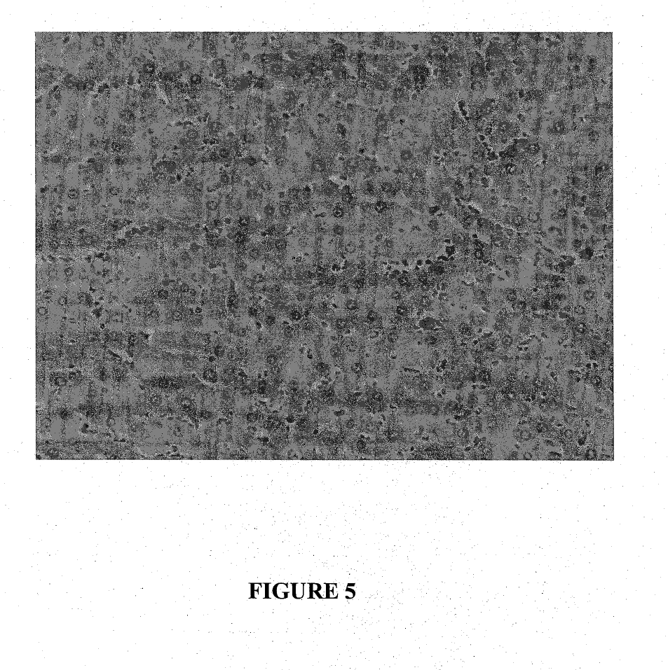

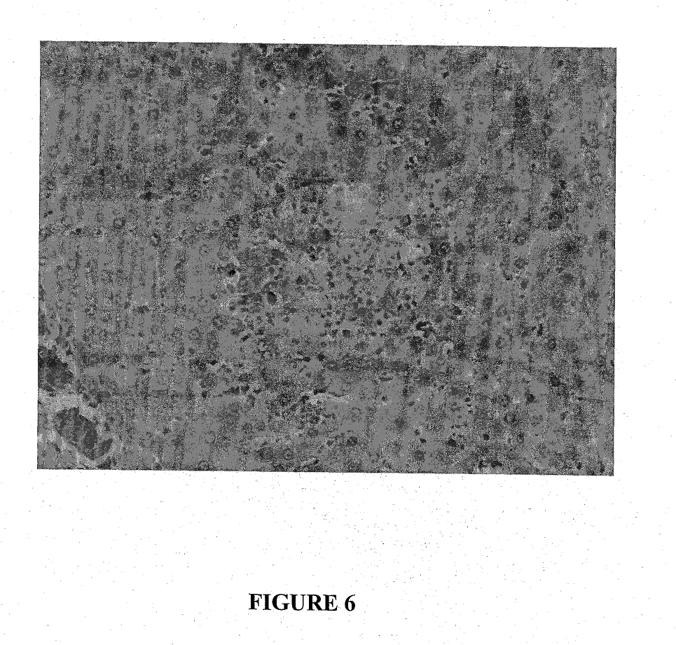

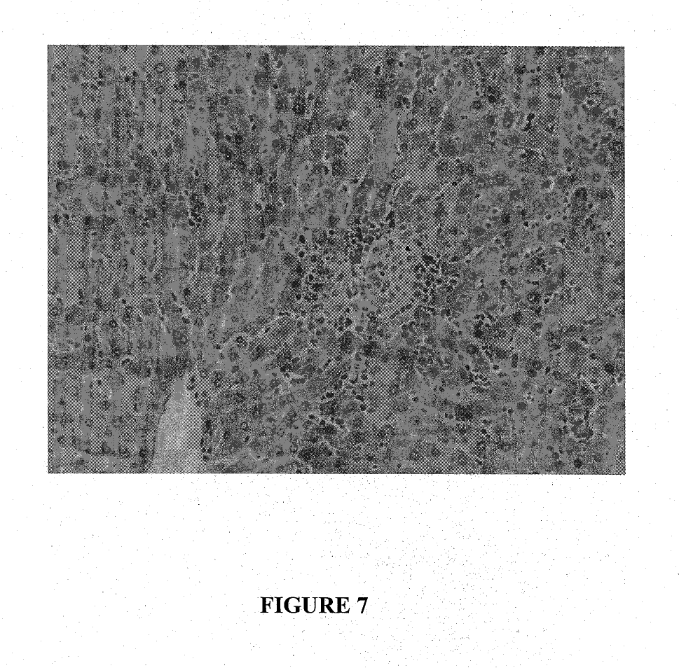

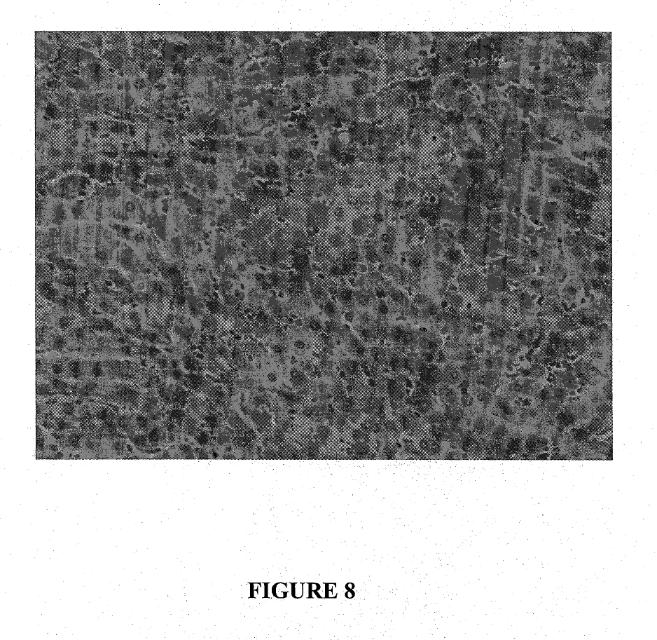

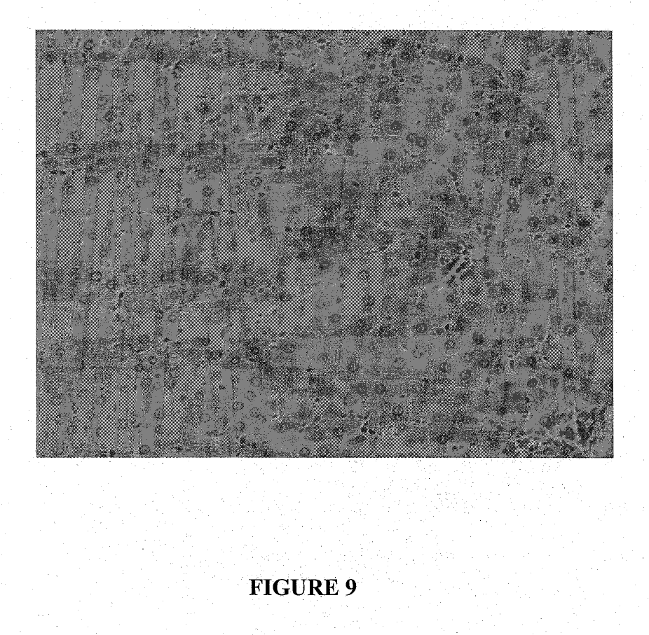

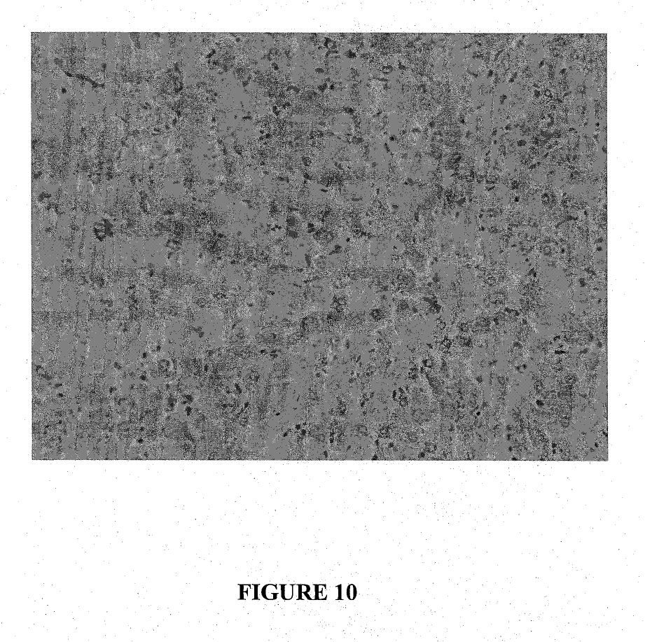

Liver micrographs are shown in FIGS. 1-10. A microscopic finding that appeared to be test article-related was the reduction of “background” inflammation in the liver of Groups 2-4 male and female rats. Multiple foci of inflammation may be found in the liver of control, barrier-maintained laboratory rats, including Sprague-Dawley rats (Spencer A J et al., 1997, Multifocal Inflammation, Liver, Rat in Monographs on Pathology of Laboratory Animals, Digestive System, Second Edition. (Jones T C, Popp J A, and Mohr U, eds.), pages 217-220. Springer-Verlag Berlin Heidelberg, New York.). Subacute hepatic inflammation (hepatitis) was present in all Group 1 (8/8) male and (6/8) female rats, but was not present in any of the Group 2 male, Group 3 female, and Group 4 male and female rats. Only one Group 3 male rat and one Group 2 female rat had minimal subacute hepatitis. This suggests that the test article was responsible for the reduction in the incidence of this idiopathic lesion in the liver of treated rats. The cause(s) of this spontaneous hepatic inflammatory lesion is unknown, but endotoxins from the intestinal tract have been proposed as a possible cause. Thus, without being limited to any particular explanation as to mechanism of action, it was concluded that the test article's antioxidant properties may have counterbalanced potential free radical or other oxidative damage in the liver induced by endotoxins.

TNF Alpha Assay Method

This test is a standard methodology for assessing the anti-inflammatory activity of compounds. Table 1 provides Percent Inhibition of LPS induced TNF-alpha in monocytes data for 18 hydroxylamine compounds of the invention.

Whole Blood TNFα:

Compounds at different concentrations (0, 1, 2.5 and 10 uM) were incubated with 100 ul freshly collected heparinized blood for 10 minutes. LPS (25 ng/mL) was added and blood was incubated at room temperature for 3 hrs. Following incubation with LPS, PBS was added (800 uL) and samples were spun for 10 minutes at 1500 g. Compounds at different concentrations (0, 1, 2.5 and 10 uM) were incubated with 100 ul freshly collected heparinized blood for 10 minutes.

TNFα protein concentrations were measured using R&D Systems high sensitivity ELISA kit.

Measurement of LPS Induced-TNFα in THP-1 Cells

THP-1 cells (0.5×10⁶ cells/mL) were incubated with the compounds (0, 1, 2.5 and 10 uM) for 3 hrs in humidified chamber, 37° C., 5% CO2, 2% FBS in RPMI medium. Cells were induced with 25 ng/mL LPS for another 3 hrs. Cells were collected and spun at 1500 g for 10 minutes. Supernatant was collected and analyzed for TNFα.

| | | Mean Percent |
|---|---|---|
| | | Inhibition of |
| Compound | | TNF-alpha using |
| (10 uM) | STRUCTURE | 5 ng/mL LPS |
| Hydroxy-4- | 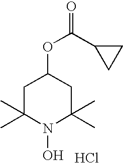 | 68 ± 6 |
| cyclopropanecarbonyloxy- | | |
| 2,2,6,6-tetramethylpiperidine | | |
| hydrochloride (Compound 1) | | |

The present invention is not limited to the embodiments described and exemplified above, but is capable of variation and modification within the scope of the appended claims.

1. A method for treating or inhibiting hepatitis in a subject in need of such treatment, comprising administering to the subject a composition comprising a pharmaceutically acceptable carrier and at least one hydroxylamine compound or ester derivative thereof in a therapeutically sufficient amount to treat hepatitis in the subject, wherein the ester derivative has the formula I: 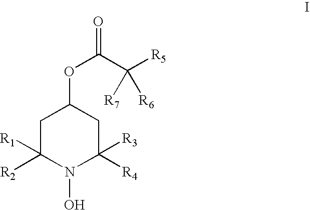
wherein:
R₁ and R₂ are, independently, H or C₁ to C₃alkyl;
R₃ and R₄ are, independently C₁ to C₃alkyl, or wherein R₁ and R₂, taken together, or
R₃ and R₄, taken together, or R₁ and R₂, taken together and R₃ and R₄ taken together, are each cycloalkyl;
R₅ is H, OH, or C₁ to C₆alkyl;
R₆ is or C₁ to C₆alkyl, alkenyl, alkynyl, or substituted alkyl or alkenyl;
R₇ is C₁ to C₆alkyl, alkenyl, alkynyl, or substituted alkyl or alkenyl; or R₆ and R₇ taken together, or R₅, R₆, and R₇ taken together, form a carbocycle having from 3 to 7 atoms in the ring or form a heterocycle having from 3 to 7 atoms in the ring.

2. The method of claim 1, wherein the hydroxylamine compound is:

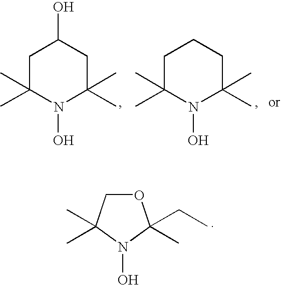

3. The method of claim 1, wherein R₁, R₂, R₃, and R₄ are each independently C₁-C₃ alkyl.

4. The method of claim 1, wherein R₁, R₂, R₃, and R₄ are ethyl.

5. The method of claim 1, wherein R₁, R₂, R₃, and R₄ are methyl.

6. The method of claim 1, wherein R₅ is H or methyl, R₆ is methyl substituted with benzyloxy or C₁-C₆ alkoxy, and R₇ is methyl.

7. The method of claim 6, wherein R₅ is H or methyl, and R₆ and R₇, taken together, form a cyclopropyl group.

8. The method of claim 6 wherein: R₅, R₆ and R₇, taken together, form a furanyl group.

9. The method of claim 6 wherein: R₅ is H; and R₆ and R₇, taken together, form a tetrahydrofuranyl group.

10. The method of claim 6 wherein: R₅ is H; and R₆ and R₇, taken together, form a cyclopropyl group.

11. The method of claim 1, wherein the subject is a mammal.

12. The method of claim 11, wherein the mammal is a human.

13. The method of claim 1, further comprising administering an antioxidant to the subject.

14. The method of claim 1, further comprising administering a reducing agent to the subject.

15. The method of claim 1, wherein the hepatitis is infectious hepatitis.

16. The method of claim 15, wherein the infectious hepatitis is caused by HAV, HBV, HCV, HDV, or HEV.

17. The method of claim 1, wherein the hepatitis is noninfectious hepatitis.

18. The method of claim 17, wherein the noninfectious hepatitis is alcoholic hepatitis, toxic/drug-induced hepatitis, autoimmune hepatitis, or granulomatus hepatitis.

19. The method of claim 1, wherein the subject has cirrhosis of the liver.

20. The method of claim 1, wherein the composition is administered to achieve in the liver of the subject a hydroxylamine concentration of about 0.1 μM to about 10 mM.

21. The method of claim 1, wherein the composition is administered to achieve in the liver of the subject a hydroxylamine concentration of about 1 μM to about 5 mM.

22. The method of claim 1, wherein the composition is administered to achieve in the liver of the subject a hydroxylamine concentration of about 10 μM to about 2.5 mM.

23. The method of claim 1, wherein the composition is administered to achieve in the liver of the subject a hydroxylamine concentration of about 50 μM to about 1 mM.

24. The method of claim 1, wherein the composition is administered to achieve in the liver of the subject a hydroxylamine concentration of about 1 μM to about 100 μM.

25. The method of claim 1, further comprising administering an additional anti-inflammatory agent.

26. The method of claim 25, wherein the additional anti-inflammatory agent is a non-steroidal anti-inflammatory drug, corticosteroid, lactoferrin, or extract from Silybum marianum, Phyllanthus, glycyrrhizin, Picrorhiza kurroa, Cudrania cochinchinensis var. gerontogea, Bidens pilosa, Glossogyne tenuifolia, Artemisia capillaries, or Sargassum polycystum.
